(19) United States
(12) Patent Application Publication
(10) Pub. No.: US 2013/0165803 A1
(43) Pub. Date: Jun. 27, 2013

(54) CUFF DEVICE

(71) Applicants: Chen-Chuan Hung, Hsinchu County (TW); Hong-Dun Lin, Taipei (TW); Cheng-Tsair Yang, Miaoli County (TW); Chun-Shen Huang, New Taipei (TW); Kuo-Hung Chan, Hsinchu County (TW)

(72) Inventors: Chen-Chuan Hung, Hsinchu County (TW); Hong-Dun Lin, Taipei (TW); Cheng-Tsair Yang, Miaoli County (TW); Chun-Shen Huang, New Taipei (TW); Kuo-Hung Chan, Hsinchu County (TW)

(73) Assignee: INDUSTRIAL TECHNOLOGY RESEARCH INSTITUTE, Hsinchu (TW)

(21) Appl. No.: 13/424,730

(22) Filed: Mar. 20, 2012

(30) Foreign Application Priority Data
Dec. 23, 2011 (TW) 100148439

(51) Int. Cl. A61B 5/022

(52) U.S. Cl. 600/499

(57) ABSTRACT

A cuff device comprising a ring and a wrapping strip with first and second surfaces is mentioned. A first coupling portion and a first fixing portion are disposed on the first surface, and a second fixing portion and a wrapping portion are disposed on the second surface. The ring comprises a ring body and a second coupling portion. The ring body moves between an end and an opposite end of the wrapping strip, and the wrapping strip can pass through the ring body. The second coupling portion is disposed on the ring body and on the second surface. Wherein the second coupling portion is bonded with a first position of the first fixing portion to form an accommodating space, the first coupling portion is folded reversely at a second position and is bonded with the first fixing portion, and thereby the accommodating space has a fixed surrounding surface.

### CROSS-REFERENCE TO RELATED APPLICATIONS

This non-provisional application claims priority under 35 U.S.C. §119(a) on Patent Application No(s). 100148439 filed in Taiwan, R.O.C. on Dec. 23, 2011, the entire contents of which are hereby incorporated by reference.

### BACKGROUND

1. Technical Field The present disclosure relates to a cuff device and more particularly to a cuff device for measuring blood pressure values.

2. Related Art

A conventional blood pressure measuring device generally comprises a cuff device and a sensing device. The cuff device is used for wrapping around an arm of a subject and comprises a bladder which can be filled with air. The bladder is tightly pressed around the arm of the subject after it is filled with air so that a blood pressure value of the subject can be measured by sensing the pressure changes in the bladder using the sensing device.

However, when using the blood pressure measuring device for measuring blood pressure, the cuff device of the blood pressure measuring device is required to be wrapped around the arm by the subject per se. The tightness of the cuff device wrapped around the arm has to be adjusted every time for measuring blood pressure, therefore it is rather inconvenient and troublesome for the user who needs to measure blood pressure and monitor physical condition frequently.

Referring to FIGS. 1 to 3, they illustrate the usage of a conventional tourniquet band. As shown in FIG. 1, a cuff device is composed of a wrapping strip 10 and a ring 20. The ring 20 is tacked at one end of the wrapping strip 10. When a blood pressure measuring device is being used, the wrapping strip 10 is wrapped around the arm 30 of a subject, and another end of the wrapping strip 10 is passed through the ring 20, in order to have a length L adjusted to be suitable for wrapping around the arm 30 of the subject.

Referring to FIGS. 1 and 2, an unwrapped portion P1 of the wrapping strip 10 is folded reversely to be overlapped and combined with a wrapped portion P2 of the wrapping strip 10.

Referring to FIG. 3, when the measuring of blood pressure is finished, the unwrapped portion P1 has to be detached from the wrapped portion P2, and then the other end of the wrapping strip 10 is pulled out from the ring 20. Therefore the length L suitable for wrapping around the arm 30 of the subject is not reserved, and the length L of the wrapping strip 10 has to be adjusted for wrapping around the arm 30 every time for measuring the blood pressure, which is rather inconvenient and troublesome for the user (e.g. an elder) who needs to monitor the blood pressure condition frequently.

### SUMMARY

A cuff device comprises a wrapping strip and a ring. The wrapping strip has first surface and a second surface. A first coupling portion and a first fixing portion are disposed on the first surface, and a second fixing portion and a wrapping portion are disposed on the second surface. The ring comprises: a ring body and a second coupling portion. The ring body moves between an end and an opposite end of the wrapping strip, and the wrapping strip can pass through the ring body. The second coupling portion is disposed on the ring body and on the second surface. The second coupling portion is bonded with a first position of the first fixing portion to form an accommodating space, the first coupling portion is folded reversely at a second position and is bonded with the first fixing portion, and thereby the accommodating space has a fixed surrounding surface.

### BRIEF DESCRIPTION OF THE DRAWINGS

The present disclosure will become more fully understood from the detailed description given herein below for illustration only, and thus are not limitative of the present disclosure, and wherein:

FIG. 1 is a usage illustration of a conventional cuff device;

FIG. 2 is a usage illustration of a conventional cuff device;

FIG. 3 is a usage illustration of a conventional cuff device;

FIG. 4 is a perspective view of a cuff device according to an embodiment of the disclosure;

FIG. 5 is a usage illustration of the cuff device in FIG. 4;

FIG. 6 is a usage illustration of the cuff device in FIG. 4;

FIG. 7 is a usage illustration of the cuff device in FIG. 4;

FIG. 8 is a perspective view of the cuff device according to another embodiment of the disclosure;

FIG. 9 is a perspective view of the cuff device in FIG. 8;

FIG. 10 is a system block diagram of a sensor; and

FIG. 11 is another system block diagram of the sensor.

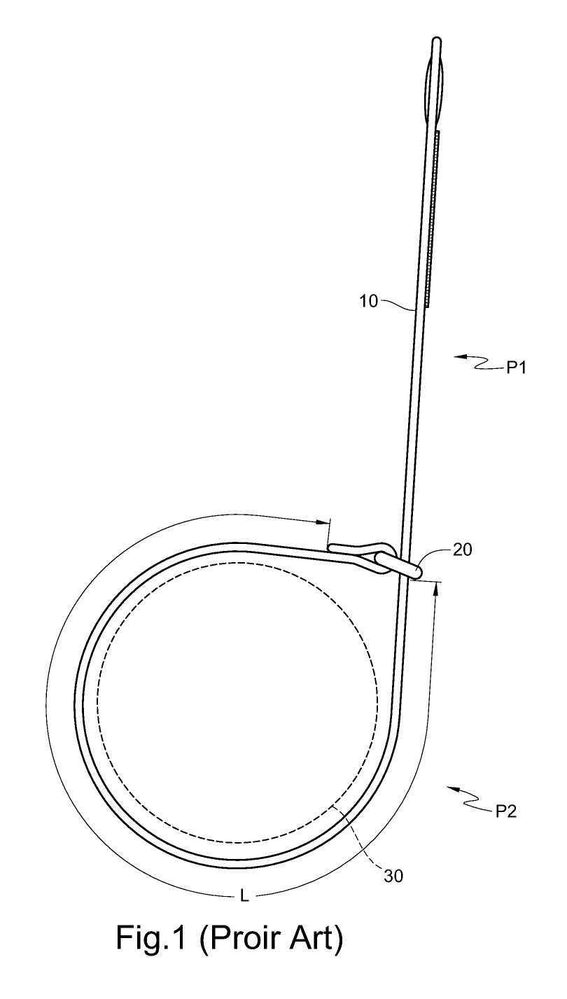

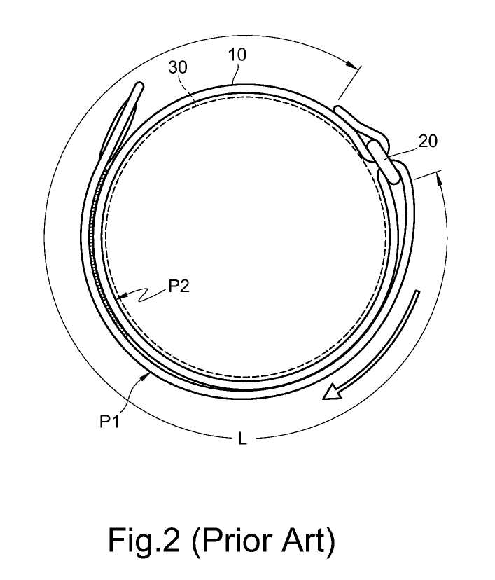

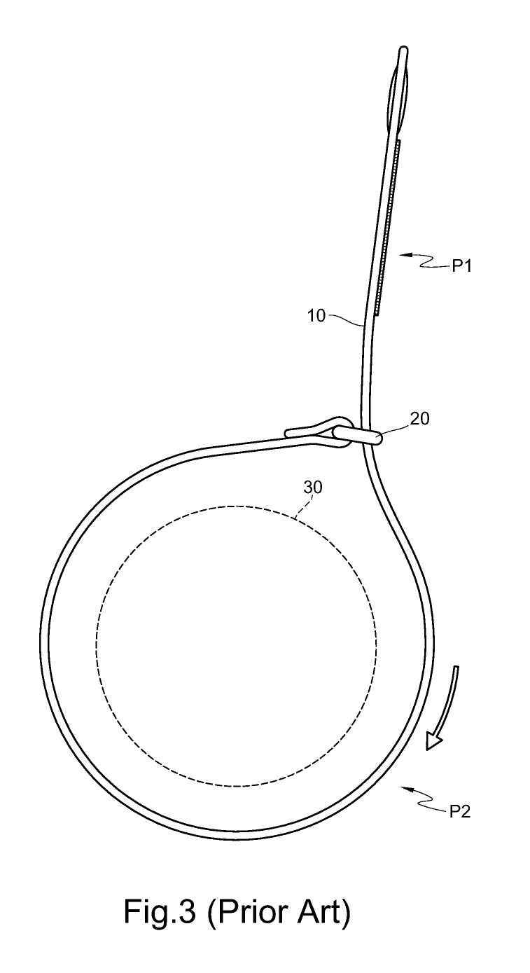

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

Some embodiment of the disclosure provides a cuff device to solve the conventional technical problems. The cuff device has a ring which can be moved freely on a wrapping strip and can be used for forming a fixed wrapping length.

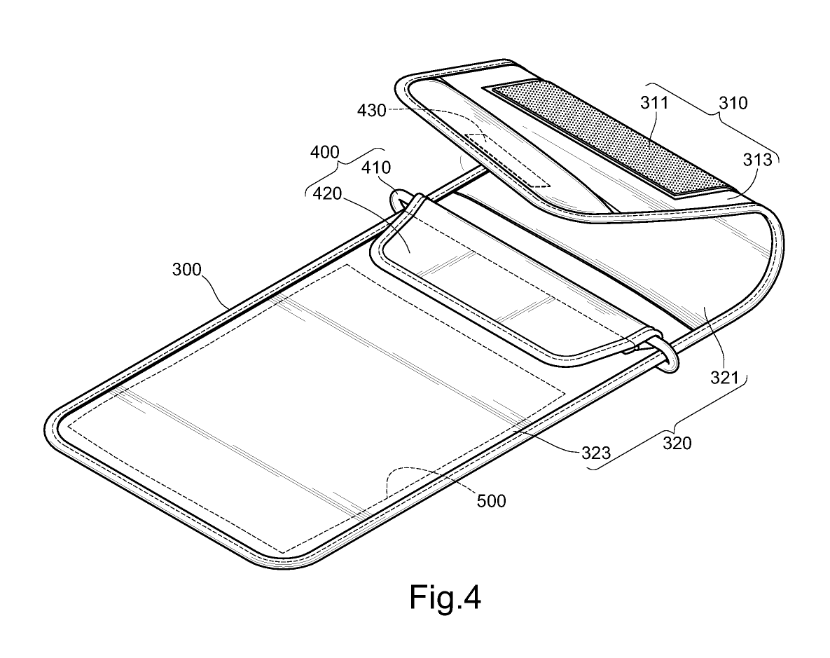

Referring to FIG. 4, which is a perspective view of the cuff device according to an embodiment of the disclosure. The cuff device comprises a wrapping strip 300 and a ring 400. The wrapping strip 300 has a first surface 310 and a second surface 320. A first coupling portion 311 and a first fixing portion 313 are disposed on the first surface 310; a second fixing portion 321 and a wrapping portion 323 are disposed on the second surface 320. The ring 400 comprises a ring body 410 and a second coupling portion 420. The wrapping strip 300 passes through the ring body 410 and the ring body 410 can be moved between an end and an opposite end of the wrapping strip 300. The second coupling portion 420 is disposed on the ring body 410 and it is also located on the second surface 320.

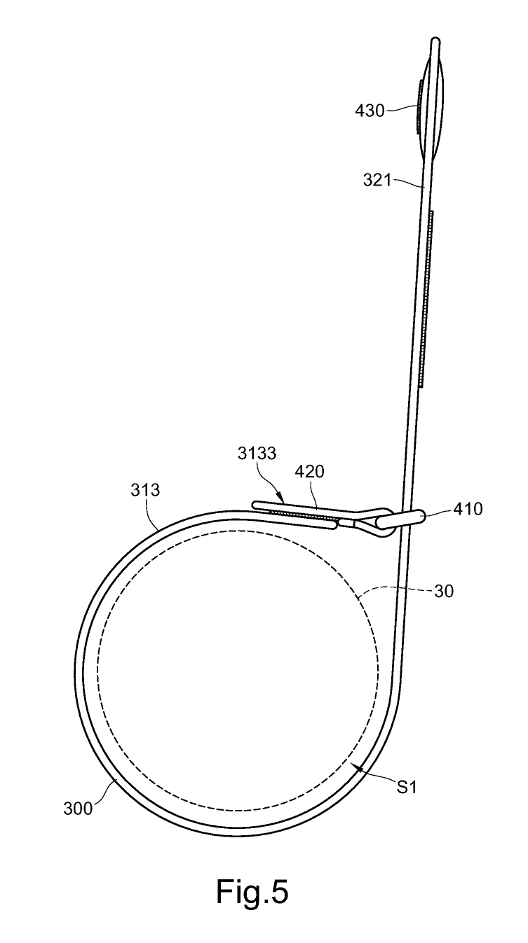

Referring to FIGS. 4 and 5, according to the abovementioned structures, an accommodating space S1 is formed when the second coupling portion 420 is bonded with a first position 3133 of the first fixing portion 313, and the size of the accommodating space S1 can be controlled by moving the ring body 410 on the wrapping strip 300. The first position 3133 is disposed on the end of the wrapping strip 300 and a third coupling portion 430 is disposed on the other end of the wrapping strip 300. The third coupling portion 430 can be folded reversely to be bonded with the second fixing portion 321 of the second surface 320, and the ring 400 can be prevented from coming off from the other end of the wrapping strip 300 by the combined third coupling portion 430.

The first coupling portion 311 and the second coupling portion 420 in this embodiment are Velcro, and the first fixing portion 313 and the second fixing portion 321 are made of woolen fabric; therefore the first coupling portion 311 can be adhered to the first fixing portion 313 or the second fixing portion 321, and the second coupling portion 420 can be adhered to the first fixing portion 313 or the second fixing portion 321, but the disclosure is not limited to them. In other embodiments of the disclosure, the first coupling portion 311 and the second coupling portion 420 can be buttons, magnets or engaging elements, and the first fixing portion 313 and the second fixing portion 321 can be button holes, magnets or engaging elements in corresponding to the first coupling portion 311 and the second coupling portion 420.

Referring to FIGS. 5 to 7, they are usage illustrations of the cuff device in FIG. 4. As shown in FIG. 5, the accommodating space S1 is formed when the second coupling portion 420 is bonded with the first position 3133 of the first fixing portion 313. In this embodiment, the accommodating space S1 is used for accommodating an arm 30, but the disclosure is not limited to it. In other embodiments of the disclosure, the accommodating space S1 can be used for accommodating, for examples, wrists, fingers or any limbs.

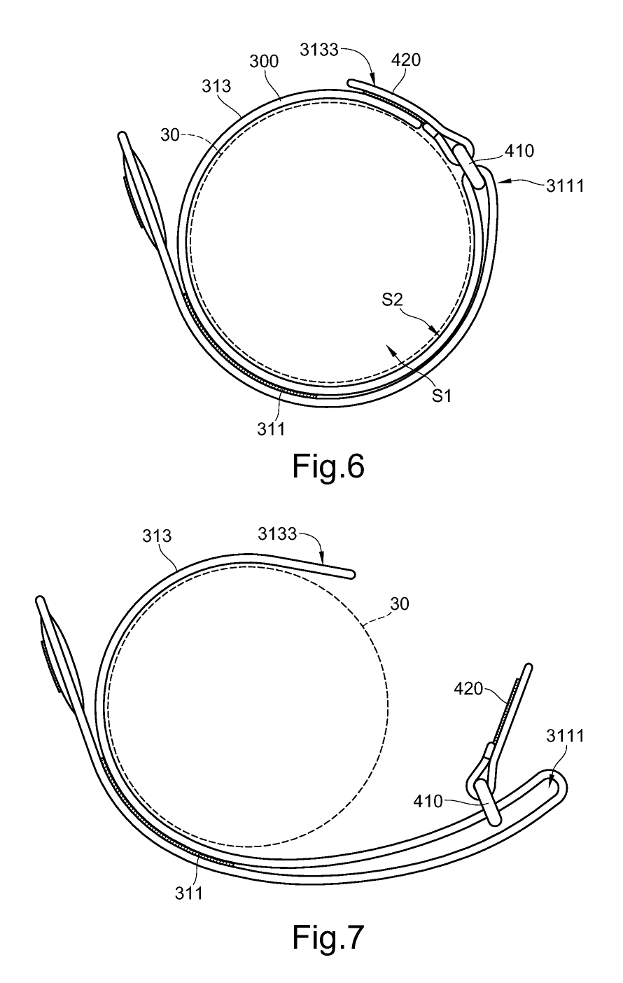

Referring to FIG. 6, a fixed surrounding surface S2 is formed for the accommodating space S1 when the first coupling portion 311 is folded reversely at a second position 3111 to bond with the first fixing portion 313. When the cuff device of this embodiment is used for measuring blood pressure, the accommodating space S1 formed by the fixed surrounding surface S2 is roughly a space required to be formed for the wrapping strip 300 for measuring the blood pressure of the arm 30. A position of the reversely folded second position 3111 can be adjusted based on the circumference of the arm 30. Referring to FIGS. 5 and 6, after the second coupling portion 420 is bonded with the first position 3133 of the first fixing portion 313, the size of the accommodating space S1 can be adjusted by moving the ring body 410 on the wrapping strip 300. When the accommodating space S1 is adjusted to have a most suitable size, the wrapping strip 300 can be folded reversely to bond the first coupling portion 311 with the first fixing portion 313, the position where the wrapping strip 300 is folded reversely is the second position 3111.

Referring to FIG. 7, the second coupling portion 420 is detached from the first position 3133 of the first fixing portion 313. Thereby, when the wrapping strip 300 is applied to similar circumference of arms next time, the fixed surrounding surface S2 formed by bonded the second coupling portion 420 with the first position 3133 of the first fixing portion 313, without the adjusting of the second position 3111 again. Therefore, each time the cuff device of this embodiment has to be applied to similar circumferences of arms, simply have the coupling portion 420 combined with or detached from the first position 3133 of the first fixing portion 313 in order to have a wrapping extent speedily adjusted for the cuff device as required by the user.

Referring to FIG. 4, when the cuff device of this embodiment is applied in measuring blood pressure, the wrapping strip 300 can further comprise a bladder 500 disposed between the wrapping portion 323 and the first surface 310. When the cuff device of this embodiment is used for measuring blood pressure, the bladder 500 is filled with air so that a sensing device externally connected with the cuff device can sense the pressure changes of the bladder 500, to achieve the effect of blood pressure measuring. The sensing device can be any devices for sensing the pressure changes of the bladder 500, for examples, an electronic sensor or a conventional mercury sensor.

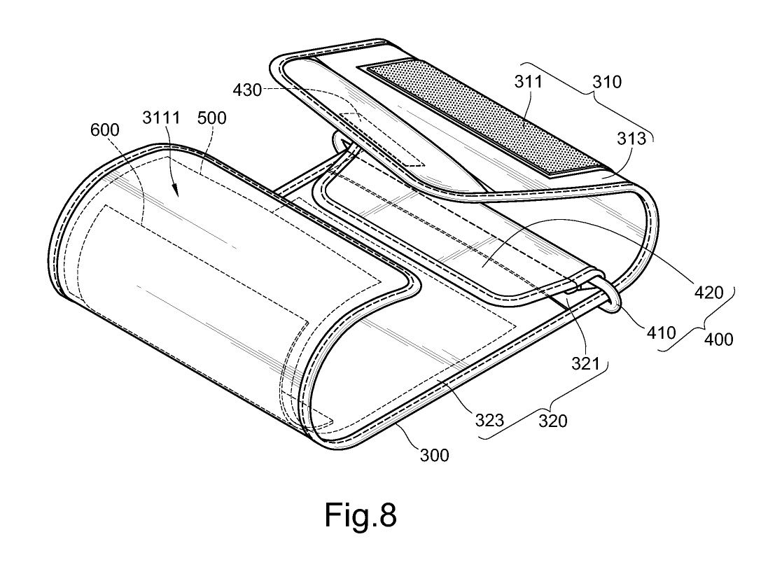

Referring to FIG. 8, which is a perspective view of the cuff device according to another of the disclosure. The difference between the embodiments associate with FIG. 4 and FIG. 8 lies in that: the embodiment of FIG. 8 further comprises a plastic plate 600 which is disposed between the wrapping portion 323 and the first fixing portion 313. In the design process of the actual product, the plastic plate 600 can be disposed inside the bladder 500. The plastic plate 600 allows the wrapping portion 323 to be formed in a wrapping shape. When this embodiment is applied in measuring blood pressure, the wrapping shape can be designed as an arc shape so that the arm 30 of the user can be placed in the space of the arc shape speedily. Furthermore, the arc shape allows the user to bond the second coupling portion 420 of the ring 400 with the first position 3111 of the first surface 310 more easily.

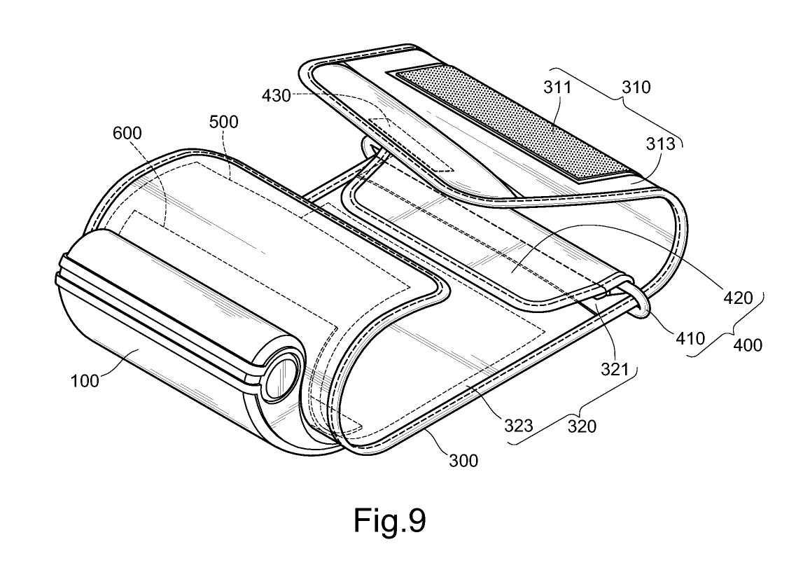

Referring to FIG. 9, which is an illustration of an electronic sensor being applied in the cuff device according to a third embodiment of the disclosure. The cuff device of the third embodiment further comprises a sensor 100 which is disposed on the first fixing portion 313 and is within the wrapping portion 323 correspondingly, therefore the sensor 100 can sense the blood pressure by the bladder 500 disposed between the wrapping portion 323 and the first surface 310.

In this embodiment, a plastic plate 600 can be further included inside the bladder 500, and the plastic plate 600 is disposed between the wrapping portion 323 and the first fixing portion 313. The sensor 100 is supported by the plastic plate 600 using the first fixing portion 313, and therefore the sensor 100 can be steadily disposed on the first fixing portion 313. Furthermore, the plastic plate 600 allows the wrapping portion 323 to have different wrapping shapes based on the requirements of application.

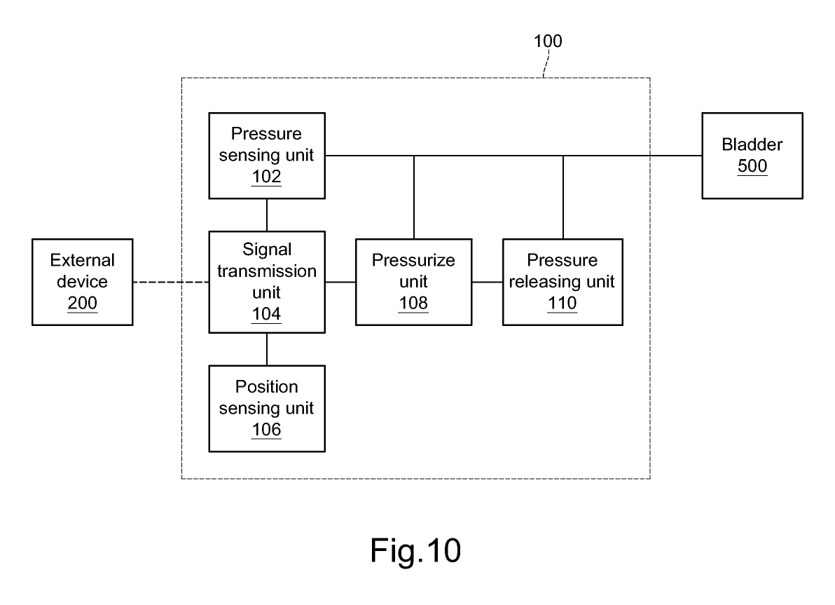

In this embodiment, the sensor 100 can be applied in different forms based on the practical requirements. Referring to FIG. 10, which is a circuit block diagram of the sensor 100. The sensor 100 at least comprises a pressure sensing unit 102 for measuring the blood pressure changes and generating blood-pressure changes information, but the disclosure is not limited to it. The sensor 100 of this embodiment further comprises a signal transmission unit 104, a position sensing unit 106, a pressurize unit 108 and a pressure releasing unit 110. The signal transmission unit 104 is coupled to the pressure sensing unit 102, and the signal transmission unit 104 is used for transmitting the blood-pressure changes information sensed by the pressure sensing unit 102 to an external device 200. The position sensing unit 106 is coupled to the signal transmission unit 104 for sensing the movement of the cuff device and generating a movement signal, and then the movement signal is transmitted to the signal transmission unit 104. The position sensing unit 106 in this embodiment can be a gyroscope or a transducer, but it is not limited to them. The movement signal is transmitted to the external device 200 by the signal transmission unit 104, so the user may be informed the movements of the arm which may cause inaccurate measuring results during blood pressure measuring.

The pressurize unit 108 is coupled to the signal transmission unit 104 and the pressure sensing unit 102, for filling the bladder 500 with air so that the data of blood pressure changes can be sensed by the pressure sensing unit 102 through the pressure changes of the bladder 500. The pressure releasing unit 110 is coupled to the pressurize unit 108 and the pressure sensing unit 102, for releasing the air of the bladder 500. In this embodiment, the external device 200 is used for controlling the pressurize unit 108 to fill the bladder 500 with air and used for controlling the pressure releasing unit 110 to release the air in the bladder 500. The external device 200 can be a tablet computer, a home computer or a smart phone. In addition to controlling the pressurize unit 108 and the pressure releasing unit 110 are, the external device 200 also can be use for storing the blood pressure measuring data, to ensure the privacy of the user. In other embodiments of the disclosure, the air-filling process of the pressurize unit 108 and the air-releasing process of the pressure releasing unit 110 can be implemented by the sensor 100 directly.

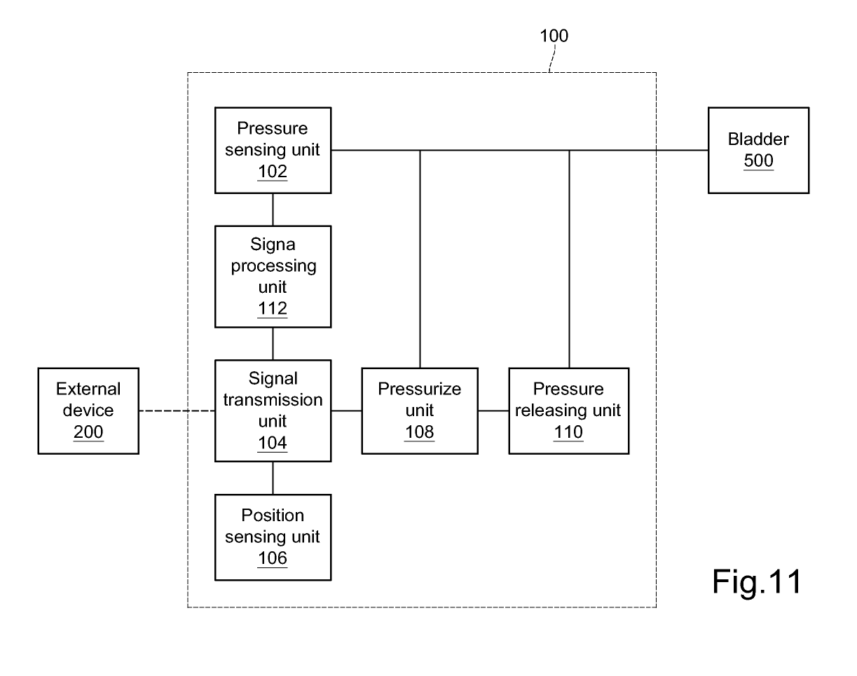

Referring to FIG. 11, which is another circuit block diagram of the sensor 100. The sensor 100 further comprises a signal processing unit 112 which is coupled between the pressure sensing unit 102 and the signal transmission unit 104. The signal processing unit 112 is used for processing the blood-pressure changes information sensed by the pressure sensing unit 102 in order to obtain blood pressure values, and the blood pressure values are transmitted to the external device 200 through the signal transmission unit 104. Furthermore, if abnormal heartbeat or hand shaking is occurred before blood pressure measuring, a corresponding warning message is generated and sent to the signal transmission unit 104 by the signal processing unit 112, and the warning message is then transmitted to the external device 200 by the signal transmission unit 104 in order to remind the user that an arrhythmia or a fault is occurred during blood pressure measuring.

The cuff device of the disclosure comprises a ring which can be moved backwards and forwards on the wrapping strip, and the ring comprises the coupling element (i.e. the second coupling portion in the embodiments) which can be bonded on the wrapping strip. Therefore, when the cuff device is applied to similar circumferences of arms, the wrapping extent required for the arm of the user can be maintained by combining the coupling element on the specific position (i.e. the first position in the embodiments) of the wrapping strip. Accordingly, each time the cuff device of the disclosure is applied to similar circumferences of arms, the extent for wrapping around the arm of the user is not required to be adjusted.

Note that the specifications relating to the above embodiments should be construed as exemplary rather than as limitative of the present invention, with many variations and modifications being readily attainable by a person of average skill in the art without departing from the spirit or scope thereof as defined by the appended claims and their legal equivalents.

What is claimed is:

1. A cuff device, comprising:
a wrapping strip having a first surface and a second surface, a first coupling portion and a first fixing portion being disposed on the first surface, a second fixing portion and a wrapping portion being disposed on the second surface; and
a ring, comprising:
a ring body, being adapted to move between an end and an opposite end of the wrapping strip, wherein the wrapping strip passes through the ring body; and
a second coupling portion being disposed on the ring body and being located on the second surface;
wherein the second coupling portion is bonded with a first position of the first fixing portion to form an accommodating space, the first coupling portion is folded reversely at a second position and is bonded with the first fixing portion, and thereby the accommodating space has a fixed surrounding surface.

2. The cuff device as claimed in claim 1, further comprising a plastic plate being disposed between the wrapping portion and the first fixing portion, the plastic plate is capable of forming a wrapping shape of the wrapping portion.

3. The cuff device as claimed in claim 1, wherein the first coupling portion and the second coupling portion are made of Velcro, and the first fixing portion and the second fixing portion are made of woolen fabric.

4. The cuff device as claimed in claim 1, further comprising a sensor disposed on the first fixing portion and within the wrapping portion.

5. The cuff device as claimed in claim 4, wherein the sensor further comprises a pressure sensing unit being adapted to measure the blood pressure changes and being adapted to generate a blood-pressure changes information.

6. The cuff device as claimed in claim 5, wherein the sensor further comprises a signal transmission unit coupled to the pressure sensing unit and being adapted to transmit the blood-pressure changes information sensed by the pressure sensing unit to an external device.

7. The cuff device as claimed in claim 6, wherein the sensor further comprises a position sensing unit coupled to the signal transmission unit and being adapted to sense the movement of the cuff device and generate a movement signal to be transmitted to the signal transmission unit.

8. The cuff device as claimed in claim 7, wherein the sensor further comprises a signal processing unit coupled with the pressure sensing unit and the signal transmission unit, the signal processing unit being adapted to process the blood-pressure changes information, and the processed blood-pressure changes information is transmitted to the external device through the signal transmission unit.
